(19) United States
(12) Patent Application Publication
(10) Pub. No.: US 2022/0168443 A1
(43) Pub. Date: Jun. 2, 2022

(54) Engineered nono-lyposomes for a targeted therapy of atherosclerosis and preparation process thereof

(71) Applicant: Università degli Studi di Genova, Genova (IT)

(72) Inventors: Patrizia Perego, Genova (IT); Domenico Palombo, Genova (IT); Pier Francesco Ferrari, Genova (IT); Roberta Campardelli, Genova (IT); Giovanni Pratesi, Roma (IT)

(21) Appl. No.: 17/616,951

(22) PCT Filed: Jun. 9, 2020
(86) PCT No.: PCT/IB2020/055404

§ 371 Date: Dec. 6, 2021

(30) Foreign Application Priority Data
Jun. 12, 2019 (IT) 102019000008745

(51) Int. Cl. A61K 47/69; A61K 9/127; A61K 47/68

(52) U.S. Cl. CPC A61K 47/6913; A61K 9/1278; A61K 47/6843; A61K 9/1271

(57) ABSTRACT

Engineered nano-liposomes (immuno-nanoliposomes) and their preparation process, for use as a treatment in atherosclerosis therapy, containing therapeutic mono-clonal antibodies and having poly-anions and/or poly-cations on the external surface, to which monoclonal antibodies specific for atheromatous plaques capable of guiding said nano-liposomes are grafted.

The present invention relates to engineered nano-liposomes, also called immuno-nano-liposomes, and their preparation process, for a targeted therapy of atherosclerosis.

Atherosclerosis is a multifactorial pathology always accompanied by an inflammatory process, from the moment of plaque onset to that of thrombus formation. The plaques originate from the accumulation of low density lipoproteins (LDL) in the sub-endothelial layer of the arteries where a process of abnormal proliferation of muscle cells, impaired leukocyte migration and macrophage activation is triggered. In the thus activated macrophages, several transcription factors induce the expression of proteins involved in the inflammatory process, such as the tumor necrosis factor α, the metalloproteases of the matrix and various interleukins. Although a complete block of these factors has positive implications in atherosclerosis, their activity is still fundamental in several molecular mechanisms involved in the immune system and beyond. Hence the need for targeted inhibition of these transcription factors.

To date there are different pharmacological approaches to atherosclerosis therapy. These include: (1) HMG-CoA reductase (statin) enzyme inhibitors, (2) cholesterol absorption inhibitors and (3) fibrates. These classes of molecules are made up of drugs that target biochemical systems involved in the metabolism of cholesterol, but none of these is able to attack the atherosclerotic plaque directly from the inside, acting on the molecular mechanisms that are triggered during the formation of the plaque itself.

We now with the use of drug delivery nanosystems can achieve a targeted inhibition of the transcription factors specified above.

The object of the present invention are engineered nano-liposomes (immuno-nanoliposomes), unilamellar and/or multilamellar having a diameter preferably between 10 and 200 nm, which can be used as a treatment in the treatment of atherosclerosis, characterized by the fact that they contain a therapeutic monoclonal antibodies, that is active against the atheromatous plaque, and to present on the external surface of said nano-liposomes of the poly-anions and/or poly-cations.

Therapeutic monoclonal antibodies can be chosen preferably from those capable of blocking molecules involved in the transduction of pro-inflammatory signals or commercial antibodies with the ability to reduce cardiovascular events, such as, for example, Canakinumab developed by Novartis.

In particular, the therapeutic monoclonal antibodies are imprisoned in the nano-liposomes and the poly-anions and/or poly-cations are anchored to the surface of said nano-liposomes, where, to the terminal attachment of the said poly-anions and/or polycations are grafted monoclonal antibodies specific for atheromatous plaques capable of guiding said engineered nano-liposomes at the target tissue level.

The poly-anions and/or the poly-cations to be used are preferably selected from polyethylene glycol (PEG), hydrophilic polymers and proteins.

The choice of antibodies to be grafted to the terminal attachment of said poly-anions and/or polycations falls preferably on antibodies directed against specific epitopes of oxidized low density lipoproteins present in high concentration at the level of the atherosclerotic plaque, as well as for protein domains of the fibronectin present at the level of atheromatous plaque as well as antibodies directed against epitopes of proteins expressed exclusively at the level of atheromatous plaques in the florid phase.

A further object of the present invention is represented by a pharmaceutical composition for systemic use, in particular for the treatment in the cure of atherosclerosis, containing the engineered nanoliposomes described above in mixture with excipients and/or diluents and/or delivery systems.

Therefore the novelty of the invention is represented on the one hand by the definition of a drug delivery system for one of the most important pathologies of the vascular system, and on the other by the treatment of atherosclerosis with therapeutic antibodies.

In the last decade, more and more attention has been paid to drug delivery systems as they allow for a site-specific release of the encapsulated molecules that can be modulated over time, decreasing the unwanted effects of classic therapy.

The encapsulation of bioactive molecules, such as therapeutic antibodies, has the advantage of improving their bioavailability and pharmacokinetics. Liposomes, thanks to their low costs, their high biocompatibility and their ability to encapsulate even chemically different molecules, represent ideal encapsulation systems for the synthesis of nanoparticles to be used in targeted therapy protocols.

Furthermore, nanoliposomes are an excellent substrate for the electrostatic self-assembly of polyanions/polycations on their surfaces. These allow to stabilize the nanoparticles themselves, to modulate the release of the encapsulated agent and to allow the immobilization of antibodies on their surface.

Since atherosclerotic plaques have specific molecular markers, their epitope can represent an excellent target for guiding a nanoparticle system conjugated with a specific antibody, immobilized on the surface of the nanoparticles themselves, at the pathological site level.

These immuno-nanoparticles will be able to modulate the inflammatory process which is one of the processes underlying vascular obstruction in the presence of atherosclerotic plaques. This invention will overcome the limitations currently associated with classical therapy.

In fact, on the whole, the expected results aim to develop an innovative therapeutic approach for the treatment of atheromatous plaques in the florid phase, avoiding the systemic approach with innovative drugs but with side effects avoidable with low dosages concentrated on the plaque.

The nanosystems at its base, or the liposomes, are easy to synthesize, very versatile, highly biocompatible.

The proposed targeted therapy completely eliminates the undesirable effects that the non-specific administration of an inhibitor of these factors could have on the entire immune system of the patient. This innovative drug delivery system allows you to overcome the disadvantages due to classic therapy, decreasing its side effects, as the pharmacologically active agent will be transported and released only at the level of the atherosclerotic plaque, in the right dosage and in the pre-defined times. A drug delivery system for the treatment of atherosclerosis, such as the one described above, appears to be a highly innovative pharmacological approach, also considering the scarce literature in this regard.

A further object of the invention is represented by the synthesis of said engineered nanoliposomes (immuno-nanoliposomes) to be used in the therapy of atherosclerosis where monoclonal therapeutic antibodies are imprisoned in the nano-liposomes and polyethylene glycol, hydrophilic polymers or proteins are anchored to the surface of the nano-liposomes.

In detail, the invention provides for the definition of a simple and economic process of encapsulation of therapeutic proteins, monoclonal antibodies directed towards molecules involved in the inflammatory process, in nanoliposomes specific for the atheromatous plaque, given the presence on their surface of an antibody that recognizes proteins expressed exclusively at this level.

The process for preparing the engineered nanoliposomes specified above essentially comprises the following steps:

a) production of nano-liposomes loaded with monoclonal antibodies active against the atheromatous plaque by hydration of lipid film;

b) surface modification of said nano-liposomes by means of poly-anions and/or poly-cations;

c) grafting of monoclonal antibodies specific for atheromatous plaques capable of guiding said engineered nano-liposomes at the target tissue level (i.e. directed against atheromatous plaques) to the terminal attachment of said poly-anions and/or polycations.

The encapsulation process carried out in step a) allows to obtain nanoliposomes that have suitable chemical-physical characteristics, release and efficacy of the encapsulated active principle and are bio- and hemocompatible.

Stage (b) concerns the surface modification of the liposomes in order to make the surface more suitable for attack by an antibody, which occurs in stage (c), highly specific for atheromatous plaques capable of guiding the nanoparticles at the target tissue level.

The production of nano-liposomes loaded with monoclonal antibodies active against the atheromatous plaque by hydration of lipid film, (stage a), is carried out using a solution at different concentrations of lipids prepared with different types of solvent, which is evaporated in order to obtain the deposition of a lipid layer, which is subjected to hydration by adding a moisturizing solution containing or consisting of water and/or saline buffers and/or physiological solutions, in which a therapeutic monoclonal antibody, i.e. active against of the atheromatous plaque, has been dispersed to be encapsulated, and is carried out under stirring until a liposomal suspension is obtained, which is subsequently subjected to ultrasonic sonication, in order to obtain a good control of the granulometry, then subjected to centrifugation and washing, preferably with water, possibly repeated preferably from one to three times, until the non-encapsulated agent is removed, finally obtaining the supernatant and the pellet.

The supernatant can be stored and possibly reused in a subsequent production cycle.

The thus obtained pellet is normally resuspended in a volume of liquid (buffer, physiological solution) and centrifuged and washed again in order to achieve the desired concentration, preferably between about 106 and 107 particles/mL, and in order to remove any traces of non-encapsulated active agent that could interfere in the following steps.

The solution is preferably prepared with a number of different lipid concentrations equal to or greater than 5 comprised between 1 and 20 mg/mL and preferably with a number of different solvents used equal to or greater than 4.

In the case of at least 4 solvents used, said solvents they are ethanol, methanol, chloroform, ethyl acetate.

The lipids can be preferably chosen among the phospholipids, in particular the derivatives of the phosphatidic acid, in which the glycerol is esterified in position 1 and 2 with fatty acids and in position 3 with orthophosphoric acid. Orthophosphoric acid, in addition to esterification with glycerol, has a second esterification with an alcohol (amino alcohol or an amino acid with alcohol group or a sugar).

Said phospholipids are preferably selected from phosphatidyl-choline, phosphatidyl-ethanolamine and phosphatidyl-inositol.

The solution prepared at different lipid concentrations with different types of solvent is evaporated preferably under reduced pressure, for a time of between 0.5 and 1.5 h, at a temperature of about 40° C. The complete evaporation of the solvent is recommended to obtain the deposition of a double layer of phospholipidic with reduced thickness.

The hydration of the lipid film preferably takes place at temperatures between 15 and 40° C., for times between 0.5 and 3 hours and under mild stirring between 200 and 600 rpm, with a moisturizing/lipid ratio of between 5 and 30.

Therapeutic monoclonal antibodies to be encapsulated, i.e. active against atheromatous plaque, are preferably antibodies capable of blocking molecules involved in the transduction of pro-inflammatory signals or commercial antibodies with the ability to reduce cardiovascular events, such as, for example, canakinumab, developed by Novartis, which is aliquoted and dissolved in the aforementioned aqueous phase in order to have a loading of the active agent.

The formed liposomes can be unilamellar and/or multilamellar (mixed liposomal preparation), with dimensions preferably comprised between 10 and 200 μm.

The ultrasonic sonication is preferably carried out for a time of between 1 and 5 minutes, with intervals of 10 to 30 seconds, and at temperatures of between 15° C. and 40° C., the delivery of the ultrasounds can possibly take place in pulsed, impulse mode on for 1 to 10 seconds, pulse off for 3 to 15 seconds.

For example, a probe, a sonicator with tip immersed directly in the suspension, with powers ranging from 30% to 70% with respect to the total output power, can be used as equipment.

The liposomes obtained after ultrasonic sonication can be unilamellar and/or multilamellar, with dimensions preferably between 10 and 200 nm and dispersion indices of 5 up to 30%.

Centrifugation (or centrifugations) of the liposomal suspension, which follows ultrasonic sonication, preferably takes place at speeds between 10000 and 24000 rpm in order to recover the liposomes produced and separate them from the external water which contains the drug which may not be encapsulated.

The surface modification of the nano-liposomes by means of poly-anions and/or poly-cations (stage b), in order to make the surface suitable for the attack of the antibody responsible for the effective targeting of the nanostructure to the target area of the treatment, preferably this takes place through a reaction step of chemical attacks (covalent bonds), chemical-physical (adsorption) and/or electrostatic attacks (non-covalent bonds), so as to anchor said poly-anions and/or poly-cations to the surface of the liposomes themselves.

The poly-anions and/or the poly-cations used are preferably selected from polyethylene glycol (PEG), hydrophilic polymers and proteins.

The choice of these antibodies may fall on antibodies directed against specific epitopes of oxidized low density lipoproteins present in high concentration at the level of atherosclerotic plaque, as well as for protein domains of fibronectin present at the level of atheromatous plaque as well as antibodies directed against epitopes of proteins expressed exclusively at the level of atheromatous plaques in the florid phase.

The grafting of monoclonal antibodies specific for atheromatous plaques, capable of guiding said engineered nano-liposomes at the target tissue level, to the terminal attack of the poly-anions and/or poli-cations (stage c) can take place by covalent or not covalent coupling.

Appropriate modifications to the terminals of the poly-anions and poly-cations allow the covalent coupling of ligands.

For the non-covalent coupling of antibodies to liposomes, antibodies and lipids functionalized with proteins or small molecules that have strong mutual affinities can be used instead.

In both cases, both for the covalent and the non-covalent attack, stage (c) is preferably carried out by incubating the modified nano-liposomes in stage (b), working with an excess of antibody, at a specific temperature (“phase-transition temperature”, depending on the type of lipid or phospholipid used) for reaction times of between 0.5 and 12 hours in order to obtain the integration of the specific target antibody to the lipid bilayer of the nanocarrier.

The suspension obtained is centrifuged and washed, at speeds preferably comprised between 10000 and 24000 rpm in order to recover the liposomes produced and separate them from the external water which contains the antibody not grafted onto the liposome surface and any reaction intermediates.

The centrifugation and washing operation can preferably be repeated from one to a maximum of three times. The antibody not bound to the surface can be recycled for new grafting phases.

A further object of the invention is represented by the use of the engineered nano-liposomes as described above as a treatment in the cure of atherosclerosis.

The invention claimed is:

1. Engineered nano-liposomes, or immuno-nanoliposomes. comprising:
monoclonal antibodies active towards an atheromatous plaque; and
polyanions and/or polycations on an outer surface of said engineered nano-liposomes.

2. The engineered nano-liposomes according to claim 1, wherein said engineered nano-liposomes are unilamellar and/or multilamellar having a diameter between 10 and 200 nm.

3. The engineered nano-liposomes according to claim 1, wherein the monoclonal antibodies active towards the atheromatous plaque are imprisoned in the engineered nano-liposomes, and wherein the polyanions and/or the polycations are anchored to the outer surface of said engineered nano-liposomes where, at a terminal attachment of said polyanions and/or said polycations, second monoclonal antibodies specific for the atheromatous plaque are grafted and adapted to drive said engineered nanoliposomes at a target tissue level.

4. The engineered nano-liposomes according to claim 1, wherein the polyanions and/or the polycations are selected from the group consisting of polyethylene glycol (PEG), hydrophilic polymers, and proteins.

5. A pharmaceutical composition for systemic use, comprising:
the engineered nano-liposomes according to claim 1 in admixture with excipients, diluents, and/or delivery systems.

6. The pharmaceutical composition according to claim 5, wherein the pharmaceutical composition is adapted to be a treatment in an atherosclerosis therapy.

7. A process for preparing the engineered nanoliposomes according to claim 1, comprising:
(a) producing the engineered nano-liposomes loaded with the monoclonal antibodies active towards the atheromatous plaque by hydrating a lipid film;
(b) surface modifying said engineered nano-liposomes with the polyanions and/or the polycations; and
(c) grafting the monoclonal antibodies active for the atheromatous plaque, so as to drive said engineered nano-liposomes at a target tissue level to a terminal attachment of said polyanions and/or said polycations.

8. The process according to claim 7, wherein producing the engineered nano-liposomes comprises using a solution with different lipid concentrations prepared with different types of solvent, wherein the solution is evaporated to obtain a deposition of one lipid layer, which is subjected to hydration by adding a hydrating solution containing water, saline buffers, and/or physiological solutions, and wherein a therapeutic monoclonal antibody has been dispersed in the solution so as to be active towards the atheromatous plaque, to be encapsulated, the solution being made under stirring until obtaining a liposomal suspension, which is subsequently subjected to ultrasound sonication, then subjected to centrifugation and washing, finally obtaining a supernatant and a pellet.

9. The process according to claim 8, wherein the pellet is resuspended until reaching a desired concentration.

10. The process according to claim 8, wherein lipids in the lipid film are phospholipids selected from the group consisting of phosphatidylcholine, phosphatidyl-ethanolamine and phosphatidyl-inositol.

11. The process according to claim 8, wherein the step of hydrating the lipid film occurs at temperatures between 15 and 40° C. with times between 0.5 and 3 hours and under stirring between 200 and 600 rpm, with a hydrating agent/lipid ratio between 5 and 30.

12. The process according to claim 8, wherein the ultrasound sonication is carried out for a time between 1 and 5 minutes, with ranges from 10 to 30 seconds, and at temperatures between 15° C. and 40° C.

13. The process according to claim 8, wherein the centrifugation of the liposomal suspension, following the ultrasound sonication, occurs at speeds between 10000 and 24000 rpm.

14. The process according to claim 7, wherein the step of surface modifying the engineered nano-liposomes with the polyanions and/or the polycations occurs through a reaction step of attachments of chemical type with covalent bonds, of chemical-physical type through adsorption, and/or of electro-static type with non-covalent bonds.

15. The process according to claim 7, wherein the monoclonal antibodies are grafted to the terminal attachment of the polyanions and/or the polycations by incubation of the engineered nano-liposomes modified in the step of surface modifying by processing with an excess of antibody, at a temperature depending on a type of lipid or phospholipid used for reaction times between 0.5 and 12 hours, followed by centrifugation and washing.

16. A method of treating a patient affected by atherosclerosis, comprising:
administering engineered nano-liposomes according to claim 1 to the patient.
